(19) United States
(12) Patent Application Publication
(10) Pub. No.: US 2007/0118914 A1
(43) Pub. Date: May 24, 2007

(54) Oncohumouse

(75) Inventors: Jacques F. Banchereau, Dallas, TX (US); Anna Karolina Palucka, Dallas, TX (US)

Correspondence Address: CHALKER FLORES, LLP, 2711 LBJ Freeway, Suite 1036, Dallas, TX, 75234, US

(73) Assignee: Baylor Research Institute, Dallas, TX (US)

(21) Appl. No.: 11/544,230

(22) Filed: Oct. 6, 2006

Related U.S. Application Data
(60) Provisional application No. 60/724,316, filed on Oct. 6, 2005.

(51) Int. Cl. A01K 67/027

(52) U.S. Cl. 800/18

(57) ABSTRACT

The present invention includes a model system and use of the same for the study, manipulation and focusing of the human immune in a mammal, e.g., a nonhuman mammal that is immune deficient with one or more human hematopoietic progenitor cells and one or more human tumor cell lines.

### CROSS-REFERENCE TO RELATED APPLICATIONS

This application claims priority to U.S. Provisional Application Ser. No. 60/724,316, filed Oct. 6, 2005, the contents of which is incorporated by reference herein in its entirety.

This invention was made with U.S. Government support under National Institutes of Health Contract Nos. RO-1 CA89440, U19 AIO57234, RO-1 CA78846 and CA85540. The government has certain rights in this invention. Without limiting the scope of the invention, its background is described in connection with mouse models.

### TECHNICAL FIELD OF THE INVENTION

The present invention relates in general to the field of mammalian model systems, and more particularly, to a model system for the evaluation of in vivo interactions between human cancer and the human immune system.

### BACKGROUND OF THE INVENTION

Mouse models of human disease, most particularly cancer, brought insights into disease pathogenesis and links between genetics and biology^{1,2}. Although many results obtained in mice do translate in the human, some do not because mice and humans differ in many aspects of the immune system biology³. For example, they differ in the pattern of Toll receptor expression on dendritic cells (DCs)⁴ and/or by the breadth of the CD1 molecules expression,⁵ the lack of KIR molecule expression on mouse natural killer cells⁶, or the expression of MHC class II antigens by endothelial cells and activated T cells³. These differences might explain in part why many successful pre-clinical immunotherapy studies in mice turn unsuccessful when put in clinical trials in humans.

Animals with Human Immune Systems. A number of transgenic animals have been developed that include heterologous human immune systems and typically a knocked out endogenous immune system. Mice are a preferred species of nonhuman animal. For the production of humanized antibodies, transgenic mice have been made with a human immunoglobulin gene miniloci encoding a unrearranged human heavy (μ and γ) and κ light chain immunoglobulin sequences (sometimes referred to as HuMAb mice). More recently, mice with with targeted mutations that inactivate the endogenous μ and γ chain loci (see, e.g., Lonberg, et al. (1994) Nature 368(6474): 856-859 and U.S. Pat. No. 5,770,429) have found widespread use. These mice exhibit reduced or no expression of mouse IgM or κ, and in response to immunization, the introduced human heavy and light chain transgenes undergo class switching, gene rearrangements and somatic mutation to generate high affinity human IgG monoclonal (Harding and Lonberg (1995) Ann. N.Y. Acad. Sci 764:536-546; Taylor, L., et al. (1992) Nucleic Acids Research 20:6287-6295; Chen, J., et al. (1993) International Immunology 5:647-656; Tuaillon, et al. (1993) Proc. Natl. Acad. Sci USA 90:3720-3724; Choi, et al. (1993) Nature Genetics 4:117-123; Chen, J. et al. (1993) EMBO J. 12:821-830; Lonberg et al., (1994) Nature 368(6474): 856-859; Lonberg, N. (1994), Fishwild, D. et al. (1996) Nature Biotechnology 14:845-851).

Other examples of the use of mice for humanized studies include, e.g., U.S. Pat. Nos. 5,625,126; 5,770,429; 5,545,807; 5,939,598; and PCT published applications WO 98/24884; WO 94/25585; WO 93/1227; WO 92/22645 and WO 92/03918. Despite the development of tools for the creation of human immune systems, a model does not exist for the study of complete immune responses.

### SUMMARY OF THE INVENTION

The present inventors have found that a need exists for a pre-clinical, in vivo model for the study of the interaction of human cancer with the human immune system. Such models may be used to obtain clinical correlates and to directly establish a causal relationship between a given immune response and tumor progression/regression. Furthermore, they permit the testing of novel therapeutic approaches. With these considerations in mind a model of humanized mice⁷ in which immunodeficient nonobese diabetic/LtSz-scid/scid (NOD/SCID) mice with additional mutation in β2-microglobulin gene (NOD/SCID/β2m^{-/-}) were transplanted with human CD34⁺ hematopoietic progenitor cells (CD34⁺HPCs). It was found that these mice develop in vivo all subsets of human DCs; myeloid cells and B cells⁷. Adoptive transfer of autologous T cells permits us to analyze modulation of human T cell subsets. The OncoHumouse allows for a complete investigation of the immune response to human tumor cell lines transplanted for long-term study, for the development of specialized, for patient-specific analysis and for the development, monitoring and tracking of immunotherapy as well as customized immune response development.

Different types of human tumors from different tissues of origin may be used with the present invention, e.g., breast cancer adenocarcinoma and malignant melanoma. Patients bearing breast cancer adenocarcinoma and malignant melanoma demonstrate naturally occurring tumor-specific immunity⁸ both cellular^{9,10} and humoral.¹¹⁻¹⁴ Yet, in situ analysis of tumor samples from patients with breast cancer and melanoma demonstrates remarkably different pattern with regard to DC infiltration. Thus, breast cancer tissue is infiltrated with immature and mature DCs.¹⁵ Melanoma tumors are seldom infiltrated by DCs,¹⁶ which are immature¹⁷ and display molecules with immunosuppressive function.¹⁸

The present invention includes an in vivo model, and the use of the same, for the study of the human immune system. The in vivo model includes an immune deficient mouse made chimeric with one or more human hematopoietic progenitor cells and one or more human tumor cell lines. Examples of an immune deficient mouse includes: an immunodeficient nonobese diabetic/LtSz-scid/scid (NOD/SCID); an NOD/SCID β-2 microglobulin^{-/-} mice and the like. Human hematopoietic progenitor cells for use with the present invention may be, e.g., CD34⁺ hematopoietic progenitors, CD34⁺ hematopoietic progenitor cells contacted with G-CSF, or subsets or mixtures of human DCs, myeloid cells, B cells, autologous T cells and/or specific human T cell subsets and combinations thereof. Examples of human tumor cell lines include: breast cancer adenocarcinomas, malignant melanomas, e.g., Hs578T, MCF7, 1806, Me275, Sk-Mel24, COLO829 and combinations thereof. The immune deficient mouse may be bred and/or made immune compromised by, e.g., chemical exposure or sub-lethally irradiated.

Another embodiment of the present invention includes a pre-clinical in vivo model for the study of the interaction of human cancer with the human immune system that includes an immune deficient mouse made chimeric with one or more human hematopoietic progenitor cells and one or more human tumor cell lines. Pre-clinical studies using a human immune system may includes a nonhuman mammal that includes an immune deficient mammal with one or more human hematopoietic progenitor cells and one or more human tumor cell lines. The mammal may be a mouse, rat, rabbit, goat, pig, etc. Examples of cells for use at tumor cells in the present invention include one or more of the tumor cells listed in Table 3.

Yet another embodiment of the present invention is a method for evaluating a test compound suspected of promoting or inhibiting cancer by administering to a test mammal that includes an immune deficient chimeric mouse with one or more human hematopoietic progenitor cells and one or more human tumor cell lines with the test compound and monitoring the state of the one or more human tumor cell lines, a human immune response or both. For example, the compound affects immune cells, cancer cells, vascular cells, stromal cells, and combinations thereof. The method may also include the step of evaluating a group of compounds to identify one or more lead compounds.

Another embodiment of the present invention includes compositions and methods for identifying one or more lead compounds by administering to a chimeric immune deficient mouse that includes one or more human hematopoietic progenitor cells and one or more human tumor cell lines one or more members of a pool of test compounds; and monitoring the state of the one or more human tumor cell lines, a human immune response or both, wherein one or more lead compounds are identified from the pool of test compounds. The method may also include the step of further reducing the size of the one or more pools to identify one or more specific families of compounds that affect the human immune cells, the human cancer cells, or both.

Yet another embodiment of the invention includes a pool of test compounds isolated and purified by a method by administering to a chimeric immune deficient mouse that has one or more human hematopoietic progenitor cells and one or more human tumor cell lines one or more members of a pool of test compounds and monitoring the state of the one or more human tumor cell lines, the one or more human immune response or both, wherein one or more pools of lead compounds are identified from the pool of test compounds. The compounds may affect, e.g., immune cells, cancer cells, vascular cells, stromal cells, and combinations thereof. The mouse and/or the human hematopoietic progenitor cells, the one or more human tumor cell lines, or both, further with one or more transgenic genes. The present invention also includes one or more pools of compounds and/or compound identified by any of the methods taught herein.

Yet another embodiment of the present invention includes a method for customizing a patient immune response by making one or more chimeric oncohumice with one or more tumor cells and one or more immune cells, providing the one or more oncohumice with one or more agents that modulate the immune response, and determining the effects of the immune response against the tumor cells after exposure to the one or more agents. The one or more agents may be, e.g., antigens (T or B cell), adjuvants, superantigens, lymphokines, chemokines, cytokines and combinations thereof. The patient immune cells, the tumor cells or both may be autologous. The agents may also be a cocktail of agents that drive and/or modulate immune responses toward a Th1 response, a Th2 response, an NK response, a CTL response, signaling through Toll receptors, T cell anergy or combinations thereof.

### BRIEF DESCRIPTION OF THE DRAWINGS

For a more complete understanding of the features and advantages of the present invention, reference is now made to the detailed description of the invention along with the accompanying figures and in which:

FIGS. 1a to 1c are graphs that shows that tumor development was bi-phasic (FIG. 1a); that breast tumors developed faster than melanoma as measured by tumor size (FIG. 1b); and that the development of breast cancer tumors implanted in OncoHumouse was accelerated (FIG. 1c);

FIGS. 2a and 2b are immunofluorescence and immunohistochernistry analysis, respectively, of tissue sections from Hs578T breast cancer and Me275 melanoma primary tumors harvested at day 4 demonstrated the presence of human HLA-DR⁺ cells;

FIGS. 2c and 2d show flow cytometry analysis of single cell suspensions prepared from both Hs578T breast cancer and Me275 melanoma tumors;

FIG. 2e shows graphs that demonstrate that Hs578T breast tumors show significantly higher infiltration with human DC subsets than Me275 melanoma tumors at day 3 post-inoculation;

FIG. 3a shows microscopic immunohistochemical analysis of frozen tissue sections of the lymph nodes draining breast cancer and melanoma tumors also revealed differences in DC infiltrates;

FIG. 3b is flow cytometry analysis of single cell suspensions from lymph nodes draining breast cancer tumors demonstrated a large fraction of HLA-DR⁺ Lin⁻ cells (not shown) composed of both pDCs and mDCs;

FIG. 3c is a graph that shows that lymph nodes draining breast cancer tumors but not melanoma showed a preferential accumulation of mDCs over pDCs;

FIG. 3d is flow cytometry analysis of single cell suspensions demonstrated significantly lower DC infiltration in lymph nodes draining melanoma tumors;

FIG. 3e is a graph that shows that DCs from lymph nodes draining breast cancer tumors showed high levels of co-stimulatory molecules expression CD80, CD86 and CD40, while those from lymph nodes draining melanoma tumors showed high expression of CD40 but low expression of Cd8O and CD86;

FIG. 4a is a graph that shows that tumors regressed after injection with CD8 cells;

FIGS. 4b and 4c are graphs that show that clearance of established breast cancer tumors by adoptively transferred CD8+T cells depended on the presence of DCs when purified CD8+T cells were administered into to breast cancer tumors in OncoMouse, i.e., mouse without human DCs;

FIG. 5a and 5b are graphs that show the effect of CD4+T cells in the clearance of established tumors (Hs587T breast cancer tumors (FIG. 5a); but not Me275 melanoma (FIG. 5b);

FIG. 5c are three images that show a macroscopic comparison of tumor growth;

FIG. 5d are micrographs that show the staining patterns. for T cells;

FIG. 5e is a graph that compares tumor size upon implantation of different cell populations;

FIG. 5f is a graph that compares tumor size for after the implantation of different cell populations in accordance with the present invention;

FIG. 5g are graphs that compare the effect of treatment of MCF7 cells;

FIG. 6a are graphs that shows the lymphokine release profile of implanted immune cells;

FIG. 6b are graphs that show the lymphokine release profile of implanted DCs;

FIG. 6c are graphs that show intracellular cytokine staining;

FIG. 6d are graphs that show the effect of isolated DCs on allogeneic CD4⁺T cells cells;

FIG. 7a is a graph that shows the secretion of IL-13 on CD4⁺T cell dependent acceleration of breast cancer tumor development;

FIG. 7b is a graph that shows that CD4+T cells could be detected at a high frequency in the tumor bed by flow cytometry of intracytoplasmic staining of IL-13;

FIG. 7c is a graph that shows the expression of IL-13 for cohorts as in FIG. 7b;

FIG. 7d is a graph that shows the effect of IL-13 antagonists on tumor development.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “oncohumammal” is used to refer to non-human mammal that is immune deficient into which a human immune system has been grafted and to which a human cancer has been implanted. As will be apparent to the skilled artisan, a number of existing animals may be used as the immune deficient animal. Also, a number of methods for the non-lethal manufacturing of immune deficient animals is available, including non-lethal doses of radiation, chemical treatments, animals with one or more genetic mutations, the genetic manipulation of the mammal by the making of a transgenic, a knock-out, a conditional knock-out, a knock-in and the like. One example of an “oncohumammal” is an “oncohumouse,” in which a mouse is used as the platform for the introduction of at least a portion of a human immune system and a human tumor. The tumor may one or more primary tumors (e.g., autologous with the immune system implanted, i.e., from the same patient), one or more tumor cell clones and/or one or more tumor cell lines.

Immune Deficient Animal Hosts. Any immunodeficient mammal may be used to generate the animal models described herein. As used herein, the term “immunodeficient” is used to describe an alteration that impairs the animal's ability to mount an effective immune response. As used herein, an “effective immune response” is used to describe a human immune response in the host animal that is capable or, e.g., destroying invading pathogens such as (but not limited to) viruses, bacteria, parasites, malignant cells, and/or a xenogeneic or allogeneic transplant. One example of an immunodeficient mammal is the immunodeficient mouse referred to as a severe combined immunodeficient (SCID) mouse, which generally lacks recombinase activity that is necessary for the generation of immunoglobulin and functional T cell antigen receptors, and thus does not produce functional B and T lymphocytes.

Immune deficient mice, rats or other animals may be used, including those that are deficient as a result of a genetic defect, which may be naturally occurring or induced. For example, heterologous or homologous: nude mice, immunodeficient nonobese diabetic/LtSz-scid/scid (NOD/SCID) mice with additional mutation in β2-microglobulin gene (NOD/SCID/β2m^{-/-}), Rag 1^{-/-}, Rag 2^{-/-} mice and/or PEP^{-/-} mice, mice that have been cross-bred with these mice and have an immunocompromised background may be used for implanting or engrafting a human immune system and/or cells as described herein. The deficiency may be, for example, as a result of a genetic defect in recombination, a genetically defective thymus or a defective T-cell receptor region, NK cell defects, Toll receptor defects, Fc receptor defects, immunoglobulin rearrangement defects, defects in metabolism, combinations thereof and the like. Induced immune deficiency may be as a result of administration of an immunosuppressant, e.g. cyclosporin, NK-506, removal of the thymus, radiation and the like.

Various transgenic immune deficient mice are currently available or can be mated or cross-bred and selected in accordance with conventional techniques. Generally, the immune deficient mouse will have a defect that inhibits maturation of lymphocytes, particularly lacking the ability to rearrange immunoglobulin and/or T-cell receptor regions, Toll receptors, and the like. Female, male, castrated or uncastrated mice may be used depending on the effect of the availability of, e.g., androgens, on the course of the tumor growth. In addition to mice, immune deficient rats or similar rodents may also be employed in the practice of the invention.

As used herein, the term “compounds,” “agent(s),” “active ingredient(s),” “pharmaceutical ingredient(s),” “active agents,” “bioactive agent” are used interchangeably and defined as drugs and/or pharmaceutically active ingredients. The present invention may use or release of, for example, any of the following drugs as the pharmaceutically active agent in a pool of test compounds to isolate one or more lead compounds. A number of test compounds may be tested, isolated and purified using the methods of the present invention.

Examples of test compounds include, antitumor agents, anti-miotics, steroids, sympathomimetics, local anesthetics, antimicrobial agents, antihypertensive agents, antihypertensive diuretics, cardiotonics, coronary vasodilators, vasoconstrictors, β-blockers, antiarrhythmic agents, calcium antagonists, anti-convulsants, agents for dizziness, tranquilizers, antipsychotics, muscle relaxants, respiratory agents, non-steroidal hormones, antihormones, vitamins, herb medicines, antimuscarinic, muscarinic cholinergic blocking agents, mydriatics, psychic energizers, humoral agents, antispasmodics, antidepressant drugs, anti-diabetics, anorectic drugs, anti-allergenics, decongestants, antipyretics, antimigrane, anti-malarials, anti-ulcerative, peptides, anti-estrogen, anti-hormone agents, antiulcer agents, anesthetic agent, drugs having an action on the central nervous system or combinations thereof. Additionally, one or more of the following bioactive agents may be combined with one or more carriers and the present invention (which may itself be the carrier).

The test compounds may be found and/or isolated from a variety of custom and commercially available combinatorial libraries. In one embodiment, the pool of test compounds may include libraries of antitumor agents such as, chemotherapeutic agent is selected from the group consisting of adriamycin, 5-fluorouracil (5FU), etoposide (VP-16), camptothecin, actinomycin-D, mitomycin C, cisplatin (CDDP), doxorubicin, etoposide, verapamil, podophyllotoxin, carboplatin, procarbazine, mechlorethamine, cyclophosphamide, ifosfamide, melphalan, chlorambucil, bisulfan, nitrosurea, dactinomycin, daunorubicin, bleomycin, plicomycin, mitomycin, tamoxifen, taxol, transplatinum, vincristin, vinblastin, methotrexate, pilocarpine, mixtures and combinations thereof and the like.

Analgesic anti-inflammatory agents such as, acetaminophen, aspirin, salicylic acid, methyl salicylate, choline salicylate, glycol salicylate, 1-menthol, camphor, mefenamic acid, fluphenamic acid, indomethacin, diclofenac, alclofenac, ibuprofen, ketoprofen, naproxene, pranoprofen, fenoprofen, sulindac, fenbufen, clidanac, flurbiprofen, indoprofen, protizidic acid, fentiazac, tolmetin, tiaprofenic acid, bendazac, bufexamac, piroxicam, phenylbutazone, oxyphenbutazone, clofezone, pentazocine, mepirizole, and the like. Drugs having an action on the central nervous system, for example sedatives, hypnotics, antianxiety agents, analgesics and anesthetics, such as, chloral, buprenorphine, naloxone, haloperidol, fluphenazine, pentobarbital, phenobarbital, secobarbital, amobarbital, cydobarbital, codeine, lidocaine, tetracaine, dyclonine, dibucaine, cocaine, procaine, mepivacaine, bupivacaine, etidocaine, prilocaine, benzocaine, fentanyl, nicotine, and the like. Local anesthetics such as, benzocaine, procaine, dibucaine, lidocaine, and the like.

Antihistaminics or antiallergic agents such as, diphenhydramine, dimenhydrinate, perphenazine, triprolidine, pyrilamine, chlorcyclizine, promethazine, carbinoxamine, tripelennamine, brompheniramine, hydroxyzine, cyclizine, meclizine, clorprenaline, terfenadine, chlorpheniramine, and the like. Anti-allergenics such as, antazoline, methapyrilene, chlorpheniramine, pyrilamine, pheniramine, and the like. Decongestants such as, phenylephrine, ephedrine, naphazoline, tetrahydrozoline, and the like.

Antipyretics such as, aspirin, salicylamide, non-steroidal anti-inflammatory agents, and the like. Antimigrane agents such as, dihydroergotamine, pizotyline, and the like. Acetonide anti-inflammatory agents, such as hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone, betamethasone, ibuprophen, naproxen, fenoprofen, fenbufen, flurbiprofen, indoprofen, ketoprofen, suprofen, indomethacin, piroxicam, aspirin, salicylic acid, diflunisal, methyl salicylate, phenylbutazone, sulindac, mefenamic acid, meclofenamate sodium, tolmetin, and the like. Muscle relaxants such as, tolperisone, baclofen, dantrolene sodium, cyclobenzaprine.

Steroids such as, androgenic steroids, such as, testosterone, methyltestosterone, fluoxymesterone, estrogens such as, conjugated estrogens, esterified estrogens, estropipate, 17-β estradiol, 17-β estradiol valerate, equilin, mestranol, estrone, estriol, 17β ethinyl estradiol, diethylstilbestrol, progestational agents, such as, progesterone, 19-norprogesterone, norethindrone, norethindrone acetate, melengestrol, chlormadinone, ethisterone, medroxyprogesterone acetate, hydroxyprogesterone caproate, ethynodiol diacetate, norethynodrel, 17-α hydroxyprogesterone, dydrogesterone, dimethisterone, ethinylestrenol, norgestrel, demegestone, promegestone, megestrol acetate, and the like.

Respiratory agents such as, theophilline and β₂-adrenergic agonists, such as, albuterol, terbutaline, metaproterenol, ritodrine, carbuterol, fenoterol, quinterenol, rimiterol, solmefamol, soterenol, tetroquinol, and the like. Sympathomimetics such as, dopamine, norepinephrine, phenylpropanolamine, phenylephrine, pseudoephedrine, amphetamine, propylhexedrine, arecoline, and the like.

Antimicrobial agents including antibacterial agents, antifungal agents, antimycotic agents and antiviral agents; tetracyclines such as, oxytetracycline, penicillins, such as, ampicillin, cephalosporins such as, cefalotin, aminoglycosides, such as, kanamycin, macrolides such as, erythromycin, chloramphenicol, iodides, nitrofrantoin, nystatin, amphotericin, fradiomycin, sulfonamides, purrolnitrin, clotrimazole, miconazole chloramphenicol, sulfacetamide, sulfamethazine, sulfadiazine, sulfamerazine, sulfamethizole and sulfisoxazole; antivirals, including idoxuridine; clarithromycin; and other anti-infectives including nitrofurazone, and the like.

Antihypertensive agents such as, clonidine, α-methyldopa, reserpine, syrosingopine, rescinnamine, cinnarizine, hydrazine, prazosin, and the like. Antihypertensive diuretics such as, chlorothiazide, hydrochlorothrazide, bendoflumethazide, trichlormethiazide, furosemide, tripamide, methylclothiazide, penfluzide, hydrothiazide, spironolactone, metolazone, and the like. Cardiotonics such as, digitalis, ubidecarenone, dopamine, and the like. Coronary vasodilators such as, organic nitrates such as, nitroglycerine, isosorbitol dinitrate, erythritol tetranitrate, and pentaerythritol tetranitrate, dipyridamole, dilazep, trapidil, trimetazidine, and the like. Vasoconstrictors such as, dihydroergotamine, dihydroergotoxine, and the like. β-blockers or antiarrhythmic agents such as, timolol pindolol, propranolol, and the like. Humoral agents such as, the prostaglandins, natural and synthetic, for example PGE₁, PGE₂α, and PGF₂α, and the PGE₁ analog misoprostol. Antispasmodics such as, atropine, methantheline, papavenne, cinnamedrine, methscopolamine, and the like.

Calcium antagonists and other circulatory organ agents, such as, aptopril, diltiazem, nifedipine, nicardipine, verapamil, bencyclane, ifenprodil tartarate, molsidomine, clonidine, prazosin, and the like. Anti-convulsants such as, nitrazepam, meprobamate, phenytoin, and the like. Agents for dizziness such as, isoprenaline, betahistine, scopolamine, and the like. Tranquilizers such as, reserprine, chlorpromazine, and antianxiety benzodiazepines such as, alprazolam, chlordiazepoxide, clorazeptate, halazepam, oxazepam, prazepam, clonazepam, flurazepam, triazolam, lorazepam, diazepam, and the like. Antipsychotics such as, phenothiazines including thiopropazate, chlorpromazine, triflupromazine, mesoridazine, piperracetazine, thioridazine, acetophenazine, fluphenazine, perphenazine, trifluoperazine, and other major tranqulizers such as, chlorprathixene, thiothixene, haloperidol, bromperidol, loxapine, and molindone, as well as, those agents used at lower doses in the treatment of nausea, vomiting, and the like.

Respiratory agents such as, codeine, ephedrine, isoproterenol, dextromethorphan, orciprenaline, ipratropium bromide, cromglycic acid, and the like. Non-steroidal hormones or antihormones such as, corticotropin, oxytocin, vasopressin, salivary hormone, thyroid hormone, adrenal hormone, kallikrein, insulin, oxendolone, and the like. Vitamins such as, vitamins A, B, C, D, E and K and derivatives thereof, calciferols, mecobalamin, and the like for dermatologically use. Enzymes such as, lysozyme, urokinaze, and the like. Herb medicines or crude extracts such as, Aloe vera, and the like.

Mydriatics such as, atropine, cyclopentolate, homatropine, scopolamine, tropicamide, eucatropine, hydroxyamphetamine, and the like. Psychic energizers such as 3-(2-aminopropy)indole, 3-(2-aminobutyl)indole, and the like. Antidepressant drugs such as, isocarboxazid, phenelzine, tranylcypromine, imipramine, amitriptyline, trimipramine, doxepin, desipramine, nortriptyline, protriptyline, amoxapine, maprotiline, trazodone, and the like.

Anti-diabetics such as, insulin, and the like and anticancer drugs such as, tamoxifen, methotrexate, and the like. Anorectic drugs such as, dextroamphetamine, methamphetamine, phenylpropanolamine, fenfluramine, diethylpropion, mazindol, phentermine, and the like. Anti-malarials such as, the 4-aminoquinolines, alphaaminoquinolines, chloroquine, pyrimethamine, and the like. Anti-ulcerative agents such as, misoprostol, omeprazole, enprostil, and the like. Antiulcer agents such as, allantoin, aldioxa, alcloxa, N-methylscopolamine methylsuflate, and the like. The drugs mentioned above may be used in combination as required. Moreover, the above drugs may be used either in the free form or, if capable of forming salts, in the form of a salt with a suitable acid or base. If the drugs have a carboxyl group, their esters may be employed.

The acid mentioned above may be an organic acid, for example, methanesulfonic acid, lactic acid, tartaric acid, fumaric acid, maleic acid, acetic acid, or an inorganic acid, for example, hydrochloric acid, hydrobromic acid, phosphoric acid or sulfuric acid. The base may be an organic base, for example, ammonia, triethylamine, or an inorganic base, for example, sodium hydroxide or potassium hydroxide. The esters mentioned above may be alkyl esters, aryl esters, aralkyl esters, and the like.

Different lineages of immune-compromised mice may used in conjunction with the present invention. In one embodiment, the immune-compromised mouse may be made transgenic with one or more genes that are tumor suppressors, cytokines, enzymes, receptors, or even inducers of apoptosis. Alternatively, the second gene may be derived from an oncogene. Examples of oncogene include ras, myc, neu, raf erb, src, fms, jun, trk, ret, gsp, hst, bcl and abl. Genes may also include a tumor suppressor, the tumor suppressor may be, e.g., p53, p16, p21, MMAC1, p73, zac1, BRCAI and Rb. Other genes may tumor cytokine, the cytokine is selected from the group consisting of IL-2, IL-2, IL-3, IL-4, IL-5, IL-6, IL-7, IL-8, IL-9, IL-10, IL-11, IL-12, IL-13, IL-14, IL-15, TNF, GMCSF, 62 -interferon and γ-interferon. In other embodiments the gene may be an enzyme, e.g., cytosine deaminase, adenosine deaminase, .beta.-glucuronidase, hypoxanthine guanine phosphoribosyl transferase, galactose-1-phosphate uridyltransferase, glucocerbrosidase, glucose-6-phosphatase, thymidine kinase and lysosomal glucosidase. In other embodiments, the gene may be a receptor, e.g., CFTR, EGFR, VEGFR, IL-2 receptor and the estrogen receptor. In other embodiment, the gene may be an inducer of apoptosis, e.g., Bax, Bak, Bcl-X.sub.s, Bik, Bid, Bad, Harakiri, Ad E1B and an ICE-CED3 protease. In certain embodiments, the cells that are made transgenic and/or transfected are human cells that are implanted in the mouse.

The present invention further provides a method of enhancing the effectiveness of ionizing radiotherapy by administering, to a tumor site in a mammal, an anti-angiogenic factor protein prior to radiation therapy; and ionizing radiation, wherein the combination of anti-angiogenic factor administration and radiation is more effective than ionizing radiation alone.

The present invention also includes pools and/or leads of therapeutic compounds in, e.g., a pharmaceutically acceptable carrier or diluent. With respect to in vivo applications, the compounds identified by screening methods may be administered to the oncohumouse in a variety of ways including, for example, parenterally, orally or intraperitoneally. Parenteral administration includes administration by the following routes: intravenous, intramuscular, interstitial, intraperitoneal, intradural, epidural, intraarterial, subcutaneous, intraocular, intrasynovial, transepithelial, including transdermal, pulmonary via inhalation, opthalmic, sublingual and buccal, topical, including ophthalmic, dermal, ocular, rectal, vaginal and nasal inhalation via insufflation or nebulization.

The compounds may be orally administered, for example, with an inert diluent or with an assimilable edible carrier, they can be enclosed in hard or soft shell gelatin capsules, or they can be compressed into tablets. For oral therapeutic administration, the active compounds can be incorporated with an excipient and used in the form of ingestible tablets, buccal tablets, troches, capsules, sachets, lozenges, elixirs, suspensions, syrups, wafers, and the like. The pharmaceutical composition may include active compounds in the form of a powder or granule, a solution or suspension in an aqueous liquid or non-aqueous liquid, or in an oil-in-water or water-in-oil emulsion.

The tablets, troches, pills, capsules and the like can also contain, for example, a binder, such as gum tragacanth, acacia, corn starch or gelatin. Excipients, such as dicalcium phosphate, a disintegrating agent, such. as corn starch, potato starch, alginic acid and the like, a lubricant, such as. magnesium stearate, and a sweetening agent, such as sucrose, lactose or saccharin, or a flavoring agent may also be included. When the dosage unit form is a capsule, it may include a liquid carrier. Various other materials may be present as coatings or to otherwise modify the physical form of the dosage unit. For instance, tablets, pills, or capsules can be coated with shellac, sugar or both. A syrup or elixir may include the active compound, sucrose as a sweetening agent, methyl and propylparabens as preservatives, a dye and flavoring. Any material used in preparing any dosage unit will generally be pharmaceutically pure and substantially non-toxic. The active compound may be incorporated into sustained-release preparations and formulations.

The active compounds may be administered parenterally or intraperitoneally. Solutions of the compound as a free base or a pharmaceutically acceptable salt may be prepared in water mixed with a suitable surfactant, e.g., hydroxypropylcellulose. Dispersions can also be prepared in glycerol, liquid polyethylene glycols, and mixtures thereof, and in oils. Under ordinary conditions of storage and use, these preparations can contain a preservative and/or antioxidants to prevent the growth of microbes and/or chemical degeneration.

The pharmaceutical forms suitable for injectable use include sterile aqueous solutions or dispersions and sterile powders for the extemporaneous preparation of sterile injectable solutions or dispersions. For intravenous or other like use, the compounds are generally sterile and may be provided in liquid suspension and/or resuspended for delivery via syringe. It can be stable under the conditions of manufacture and storage and must be preserved against the contaminating action of microorganisms, such as bacteria and fungi. The carrier may be a solvent or dispersion medium containing, for example, water, ethanol, polyol (for example, glycerol, propylene glycol, and liquid polyethylene glycol, and the like), suitable mixtures thereof, and vegetable. oils. The proper fluidity may be maintained by the use of a coating, e.g., lecithin, and incorporation into a particle of the required size (in the case of a dispersion) and by the use of surfactants as is well known to the skilled artisan. The prevention of the action of microorganisms can be brought about by various antibacterial and antifungal agents, for example, parabens, chlorobutanol, phenol, sorbic acid, thimerosal, and the like. In many cases, isotonic agents, for example, sugars or sodium chloride may be used.

Sterile injectable solutions are prepared by incorporating the active compound in the required amount in the appropriate solvent with various other ingredients enumerated above, as required, followed by filtered sterilization. Generally, dispersions are prepared by incorporating various sterilized active ingredients into a sterile vehicle that includes the basic dispersion medium and any of the other ingredients from those enumerated above. In the case of sterile powders for the preparation of sterile injectable solutions, methods of preparation may include, e.g., vacuum drying, freeze-spraying, heat-vacuum and/or freeze drying techniques. Pharmaceutical compositions that are suitable for administration to the nose or buccal cavity include, e.g., powder, self-propelling and spray formulations, such as aerosols, atomizers and nebulizers.

The therapeutic compounds of this invention may be administered to a mammal alone or in combination with pharmaceutically acceptable carriers or as pharmaceutically acceptable salts, the proportion of which is determined by the solubility and chemical nature of the compound, chosen route of administration and standard pharmaceutical practice. The compositions may also include other therapeutically active compounds that are usually applied in the treatment of the diseases and disorders, e.g., cancer. Treatments using the present compounds and other therapeutically active compounds may be simultaneous or by intervals.

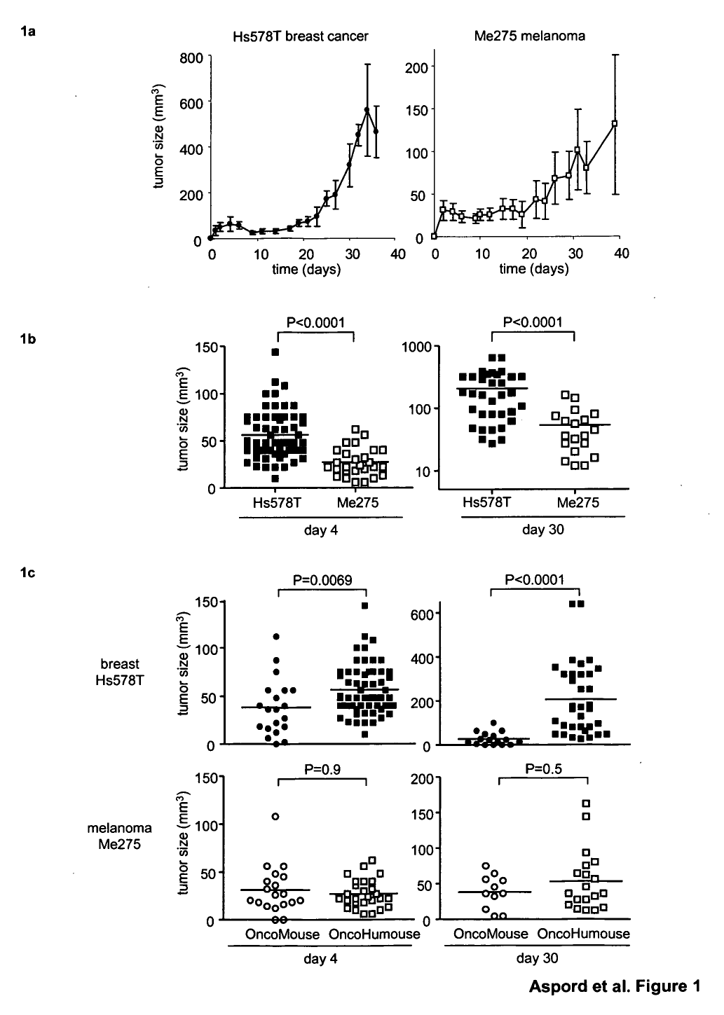

OncoHumouse develop melanoma and breast cancer tumors. Sub-lethally irradiated NOD/SCID/β2m⁻ mice were transplanted with human CD34⁺HPCs. Approximately 4 weeks later, human tumor cell lines (breast cancer or melanoma) were injected subcutaneous (s.c.) into the flank. Tumor development was bi-phasic (FIG. 1a) with a ten-day tumor establishment phase followed by a temporary decrease in the tumor volume and subsequent progression at the primary site (FIG. 1a) and the development of distant metastasis (not shown). Breast tumors developed faster than melanoma as the tumor size, 4 and 30 days after inoculation, was significantly higher (mean±SEM=56.4±3.2 vs 27.1±2.9 and 206.8±28 vs 53.3±9.8; p<0.0001, FIG. 1b). The development of breast cancer tumors implanted in OncoHumouse was accelerated when compared to those implanted in the NOD/SCID/β2m⁻ mice that did not receive human CD34⁺HPCs (OncoMouse) (56.4±3.2 vs 38.1±6.6 at day 4 and 206.8±28 vs 26.8±7.3 at day 30; FIG. 1c). That was independent of whether the mice were irradiated or not prior to tumor implantation (not shown). These results suggested that tumors, most particularly breast cancer, might utilize human immune cells for their expansion/survival.

Generation of OncoHumouse and OncoMouse. CD34⁺HPCs were obtained from healthy adult volunteers mobilized with G-CSF as previously described¹³. Mobilized peripheral blood CD34⁺ cells (2.5×10⁶/150 μl PBS) were transplanted intravenously into sublethally irradiated (12 cGy/g body weight of ¹³⁷CS γ-irradiation) NOD-SCID β₂m^{-/-} mice (Jackson). For studies with OncoMouse, NOD-SCID β₂m^{-/-} mice were sublethally irradiated (12 cGy/g body weight of ¹³⁷CS γ-irradiation) the day prior to tumor implantation. Tumor cell lines (breast cancer Hs578T, MCF7, 1806, and melanoma Me275, Sk-Me124, COLO829) were from ATCC (expect for Me275 kindly provided by Drs. J-C. Cerottini and D. Rimoldi at the Ludwig Cancer Institute in Lausanne). Cell lines were maintained in RPMI supplemented with 10% fetal bovine serum, 50 U/ml penicillin, 50 mg/ml streptomycin, 2 mM glutamine, 1 mM sodium pyruvate, amino acids, Hepes. For inoculation into Humouse, tumor cells were harvested using trypsine EDTA and washed twice in PBS. 10×10⁶ tumor cells/100 μl PBS were injected subcutaneously into the flank of the mice. Tumor size was monitored every 2-3 days. Tumor volume (ellipsoid) was calculated using the formula: (short diameter)²×long diameter/2.

Monocyte-derived dendritic cells and T cell purification. Monocyte-derived dendritic cells were generated from Ficoll-separated peripheral blood mononuclear cells. Monocytes were enriched by adherence and were cultured with granulocyte-monocyte colony-stimulating factor (100 ng/ml) and IL-4 (25 ng/ml) for 3 days. Tumor cells were killed with botulinic acid and loaded on dendritic cells for the last 48 h of the culture. To reconstitute the Humouse with T cells, autologous T cells were selected from frozen PBMC. CD4 or CD8 T cells were positively selected using magnetic selection. The purity obtained was 85% to 98% depending on the study.

Immunofluorescence and immunohistochemistry analysis. Harvested tissues were frozen in Tissue-Tek (OCT, Allegiance, McGaw, Ill.). Frozen tissues were cryosectioned on Superfrost Plus slides (Fisher scientific, Pittsburgh, Pa.) and stored at −80° C. Lymph nodes sections were -fixed with cold acetone and labeled with HLA-DR FITC and DC-LAMP pure following by anti-mouse IgG conjugated to Texas-Red and confocal imaging. Tumor sections were fixed with cold acetone and labeled with HLA-DR FITC and GP100 pure following by anti-mouse IgG conjugated to Texas-Red and confocal imaging. For immunohistochemistry, tumor sections were fixed with PFA 4%. Endogenous peroxydase activity was blocked with 0.3% H2O2. Sections were labeled with HLA-DR Ab, followed with a secondary Ab conjugated to biotin, streptavidin-peroxydase and DAB substrate. Sections were counterstained with H&E.

Flow cytometry analysis. Cell suspensions were obtained from tumor, lymph nodes and spleen after digestion of the organs with collagenase 2 mg/ml 45 min at 37° C. Bone marrow cells were also obtained. Cell suspensions were washed twice stained in PBS 2 mM EDTA 5% AB serum using the following markers: Lin, CD123, HLA-DR, CD11c, CD45, CD14, CD3, CD19, IgD, CD40, CD80, CD86 (BD).

CTL assay. Autologous T cells were purified using magnetic CD4 and CD8 positive selection and 2-5×10⁶ cells were injected intratumorally 3 times. Ten days after the last injection, CD8 T cells were purified from the tumor, lymph nodes and spleen using positive CD8 selection and restimulated 5 days in vitro with autologous monocyte-derived DC loaded with killed tumor cells and matured with CD40L in RPMI 10% AB supplemented with IL2 (10 U/ml) and IL7 (10 U/ml). CD8 T cells just stimulated once in vitro were used as control. The CTL activity was measured in a 4 h ⁵¹Cr release assay using Hs578T and K562 cell lines as targets. The percentage of killed cells was calculated using the formula: percentage of release=(cpm experiment−cpm spontaneous release)/(cpm maximum release−cpm spontaneous release)×100.

Vascularization analysis. To assess tumor vascularization, OncoHumouse were injected with FITC-lectin (150 μl at 2 mg/ml) intravenously (iv.) 10 min later mice were anesthetised and infused with PFA 4% iv. 10 μm tumor sections were fixed and stained with ToPro.

T cell cytokine production. Naive CD4 T cells were obtained from buffy coat after magnetic depletion using CD8, CD14, CD19, CD16, CD56 and glycophorine A microbeads. The cells were then sorted based on the CD4+CCR7+CD45RA+ phenotype. Depletion of NKT cells was obtained by excluding Vα24 positive CD4 T cells. DC were sorted from the tumor, draining lymph nodes, spleen and bone marrow of OncoHumouse according to HLA-DR+ Lin− phenotype and HLA-DR+Lin-CD11c+ and HLA-DR+Lin-CD123+. Naive CD4 T cells (5.10⁴/well) were cultured with DC (5.10³/well) in RPMI 1640 supplemented with 10% human AB serum. After 5 days, T cells were harvested, washed twice and resuspended at a concentration of 1.10⁶/ml. To assess cytokine production by Luminex, T cells were restimulated 16 h with PMA (50 ng/ml) and ionomycin (1 μg/ml) for 16 h. Cytokine production was analyzed in the culture supernatant by Luminex. For intracellular cytokine staining, T cells were harvested on day 6 of the culture, washed twice and restimulated 5 hours with PMA and ionomycin. Brefeldin A (10 mg/ml) was added for the last 2.5 hours. T cells were labeled with anti-CD3 Ab and intracellular cytokine staining was then performed using Abs to IL4, IL13, TNFα, IFNγ and IL2. Cells were fixed in PFA 1% and analyzed on a flow cytometer.

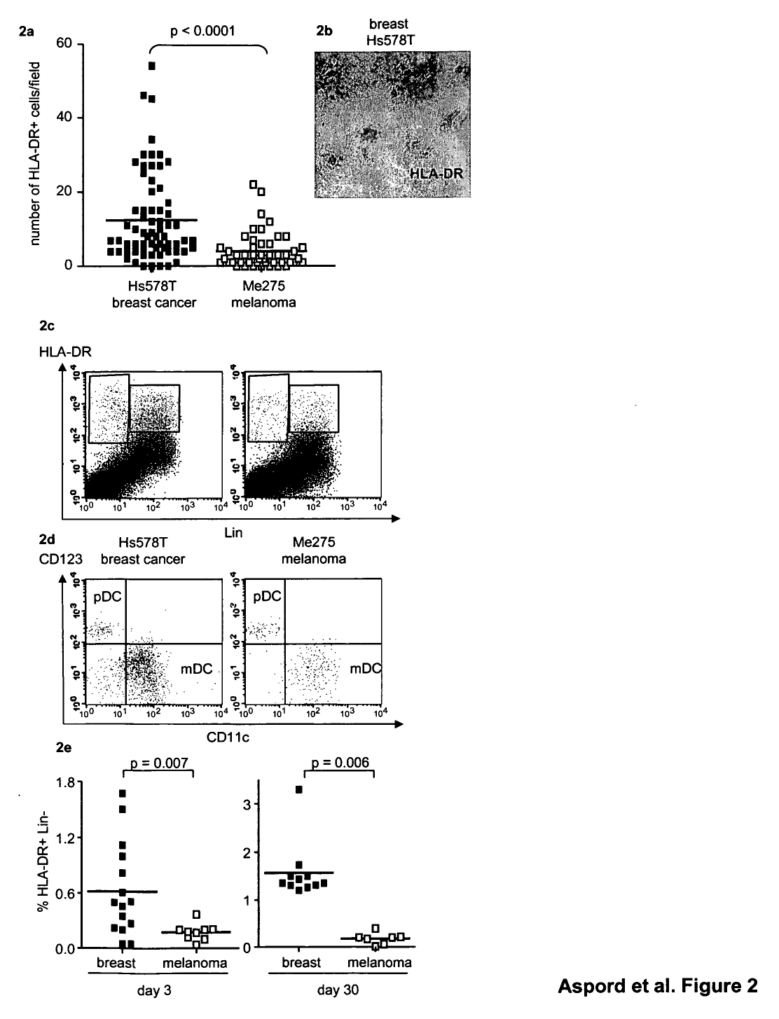

Breast cancer tumors are infiltrated with human DC subsets. Immunofluorescence and immunohistochemistry analysis of tissue sections from Hs578T breast cancer and Me275 melanoma primary tumors harvested at day 4 demonstrated the presence of human HLA-DR⁺ cells (FIG. 2). Higher numbers of human HLA-DR⁺ cells were found in sections of breast tumors than of melanoma: 12.45±1.4 and 4.1±0.7 cells per field, respectively (mean±SEM, n=69 and 47; p<0.0001, FIG. 2a and b). Similar pattern of DC infiltration was observed in metastatic tumors (not shown).

Flow cytometry analysis of single cell suspensions prepared from both Hs578T breast cancer and Me275 melanoma tumors demonstrated the presence of both HLA-DR⁺ Lineage (Lin)⁺ cells and HLA-DR⁺ Lin⁻ cells (FIG. 2c). The HLA-DR⁺ Lin⁺ cells were predominantly CD14⁺ cells that could correspond to monocytes, macrophages or interstitial DCs. The HLA-DR⁺ Lin⁻ cells contained HLA-DR⁺ CD11c⁺ myeloid DCs and HLA-DR⁺ CD123⁺ plasmacytoid DCs (FIG. 2d). Hs578T breast tumors showed significantly higher infiltration with human DC subsets than Me275 melanoma tumors already at day 3 post-inoculation (0.62±0.13 vs 0.17±0.03; p=0.007, FIG. 2e). DC infiltration further increased in Hs578T breast cancer tumors (1.57±0.18 at day 30, FIG. 2e), but not in Me275 melanoma tumors (0.18±0.05; p=0.006). The observed difference was related to the type of tumor cell line rather than difference in mice engraftment with human cells measured as the percentage of human CD45⁺ cells in bone marrow (Table 1). Three different melanoma tumors (Me275, Sk-Me124 and Colo829) showed consistently low infiltration and three different breast cancer cell tumors (Hs578T, MCF7 and 1806) showed higher infiltration with DCs (Table 1). These results indicate that breast cancer can attract and/or retain human DCs more efficiently than melanoma.

TABLE 1
Tumor cell comparison.

| type of | | time post | number | HLA-DR+ Lin− cells (%) | | | |
|---|---|---|---|---|---|---|---|
| tumor | cell line | tumor implant | of mice | tumor | draining LN | control LN | spleen |
| breast | Hs578T | 4 days | 16 | 0.74 +/− 0.17 | 15.34 +/− 3.78 | 4.75 +/− 1.71 | 21.43 +/− 1.47 |
| | MCF7 | 4 days | 4 | 0.78 +/− 0.15 | 23.7 +/− 2.6 | 4.45 +/− 0.45 | 7.8 +/− 1.94 |
| | 1806 | 4 days | 3 | 0.22 +/− 0.06 | 3 +/− 1.9 | 0.88 +/− 0.61 | 5.03 +/− 0.54 |
| | Hs578T | 30 days | 12 | 1.44 +/− 0.2 | 2.79 +/− 0.57 | 0.09 +/− 0.01 | 0.6 +/− 0 |
| | 1806 | 30 days | 8 | 0.26 +/− 0.03 | 12.45 +/− 4 | 4.1 +/− 1.01 | 2.59 +/− 1.65 |
| melanoma | Me275 | 4 days | 10 | 0.18 +/− 0.03 | 5.48 +/− 2.86 | 2.8 +/− 2.44 | 4.27 +/− 1.45 |
| | Sk-Mel24 | 4 days | 7 | 0.24 +/− 0.06 | 4.59 +/− 1.24 | 3.43 +/− 0.94 | 4.94 +/− 0.98 |
| | Me275 | 30 days | 7 | 0.19 +/− 0.04 | 2.87 +/− 0.75 | 1.86 +/− 0.59 | 1.49 +/− 1.11 |
| | COLO829 | 30 days | 4 | 0.07 +/− 0.04 | 5.16 +/− 0.68 | 3.04 +/− 1.54 | 7.08 +/− 4.02 |
| | Sk-Mel24 | 30 days | 2 | 0.02 +/− 0.00 | 3.04 +/− 1.44 | ND | 7.69 +/− 0.36 |

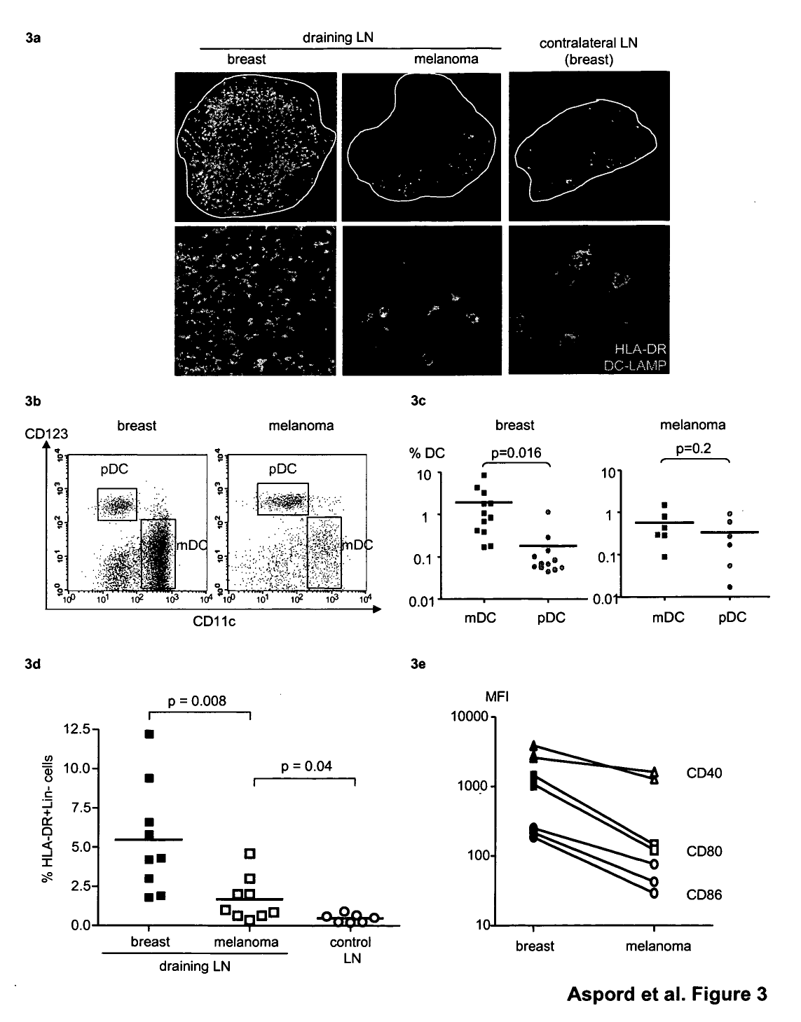

Breast cancer tumors draining lymph nodes are infiltrated with mature human DCs. Analysis of the lymph nodes draining breast cancer and melanoma tumors also revealed differences in DC infiltrates. Thus, lymph nodes draining breast cancer tumors, but not those draining melanoma tumors, were invaded with mature human DCs co-expressing HLA-DR and DC-LAMP (FIG. 3a). Flow cytometry analysis of single cell suspensions from lymph nodes draining breast cancer tumors demonstrated a large fraction of HLA-DR⁺ Lin⁻ cells (not shown) composed of both pDCs and mDCs (FIG. 3b). Both DC subsets could also be seen in lymph nodes draining melanoma tumors (FIG. 3b). Interestingly, lymph nodes draining breast cancer tumors but not melanoma showed a preferential accumulation of mDCs over pDCs (1.95±0.7 vs 0.18±0.09; p=0.02; FIG. 3c). Consistent with microscopic analysis of frozen tissue sections shown in FIG. 3a, flow cytometry analysis of single cell suspensions demonstrated significantly lower DC infiltration in lymph nodes draining melanoma tumors (5.46±1.16 for Hs578T breast cancer tumors vs 1.67±0.46 for Me275 melanoma tumors p=0.008, vs 0.47±0.1 for control contra-lateral lymph node from mice with breast cancer tumors; FIG. 3d). Furthermore, DCs from lymph nodes draining breast cancer tumors showed high levels of co-stimulatory molecules expression CD80, CD86 and CD40, while those from lymph nodes draining melanoma tumors showed high expression of CD40 but low expression of Cd80 and CD86 (FIG. 3e). These results suggest that breast cancer induced DCs to undergo maturation. This is consistent with the present inventors findings of mature DCs infiltrating peri-tumoral areas in 60% of breast cancer tissue samples¹⁵. In all studies comparing side by side different breast and melanoma tumors, i.e., Hs578T with Me275, MCF7 with Sk-Me124, or 1806 with COLO829, the percentage of DC was much higher in lymph nodes draining breast tumors than in those draining melanoma tumors (Table 2). These results indicate that different tumors attract and modulate the DCs differentially.

TABLE 2
Comparison of Hs578T v. Me275 cells.

| | | draining lymph node | | | | | |
|---|---|---|---|---|---|---|---|
| | | day 4 | | | | | |
| | | exp 1 | | exp 2 | | exp 3 | |
| | | Hs578T | Me275 | MCF7 | SK-Mel24 | 1806 | SK-Mel24 |
| % | DR+ Lin− | 8.2 +/− 2.01 | 3.3 +/− 1.3 | 27.3 +/− 0.75 | 3.1 +/− 0.05 | 3.4 +/− 1.4 | 2.6 +/− 1.15 |
| cells | DR+ CD80+ | 6 +/− 1.22 | 1 +/− 0.5 | 26.5 +/− 0.5 | 2.7 +/− 0.65 | 1.9 +/− 1.02 | 1.5 +/− 0.48 |
| | DR+ CD86+ | 5.2 +/− 1.78 | 0.14 +/− 0.05 | 6.4 +/− 0.6 | 0.13 +/− 0.07 | 1.9 +/− 1.02 | 1.6 +/− 0.47 |
| | DR+ CD40+ | 6.5 +/− 1.31 | 3.45 +/− 1.45 | 28 +/− 1 | 3.4 +/− 0.75 | 1.63 +/− 0.68 | 1.3 +/− 0.38 |
| MFI | CD80 | 1182 +/− 135 | 132 +/− 11 | 379 +/− 140 | 76 +/− 16 | 375 +/− 50 | 128 +/− 17 |
| | CD86 | 214 +/− 16 | 35 +/− 6.5 | 125 +/− 10 | 39 +/− 6 | 116 +/− 15 | 46 +/− 4.41 |
| | CD40 | 2972 +/− 290 | 1440 +/− 164 | 1692 +/− 99 | 1219 +/− 116 | 1721 +/− 169 | 1115 +/− 117 |

| | | draining lymph node | | | |
|---|---|---|---|---|---|
| | | day 30 | | | |
| | | exp 1 | | exp 2 | |
| | | Hs578T | Me275 | 1806 | COLO829 |
| % | DR+ Lin− | 1.3 +/− 0.79 | 0.2 +/− 0.15 | 23.5 +/− 2.95 | 6.2 +/− 0.52 |
| cells | DR+ CD80+ | ND | ND | 21.7 +/− 3.05 | 4.3 +/− 0.41 |
| | DR+ CD86+ | 0.9 +/− 0.42 | 0.1 +/− 0.08 | 20.1 +/− 3.15 | 4.3 +/− 0.4 |
| | DR+ CD40+ | 1.3 +/− 0.66 | 0.1 +/− 0.09 | ND | ND |
| MFI | CD80 | ND | ND | 1145 +/− 64 | 235 +/− 55 |
| | CD86 | 717 +/− 65 | 264 +/− 96 | 673 +/− 470 | 67 +/− 4.5 |
| | CD40 | 1141 +/− 46 | 923 +/− 11 | ND | ND |

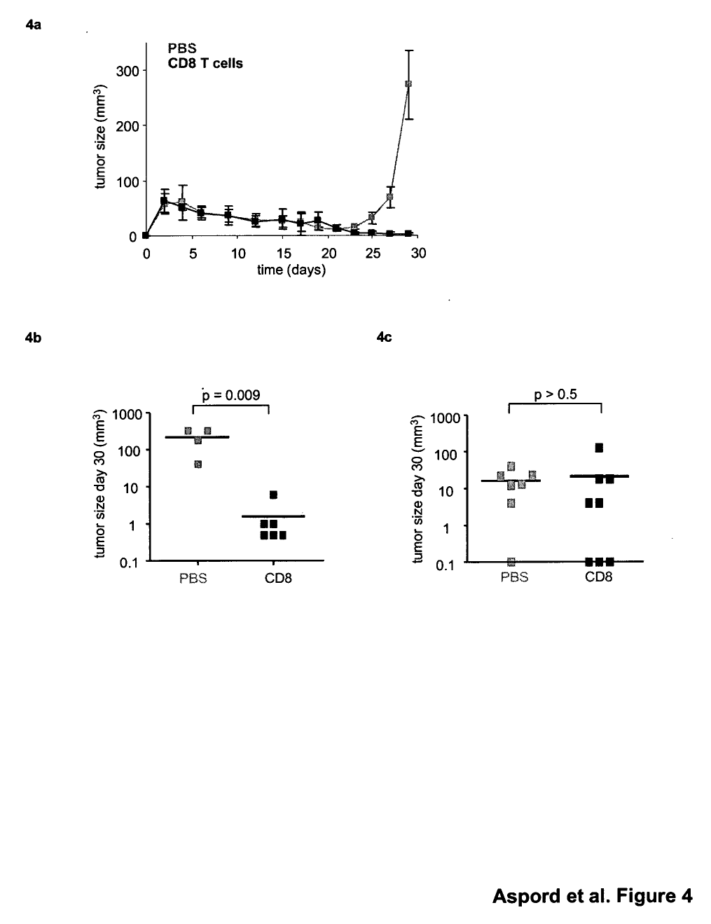

Adoptive transfer of CD4⁺T cells enhances tumor development in OncoHumice bearing breast cancer tumors. This model with adoptive transfer permits separate analysis of the interactions of human T cell subsets with human tumors. As CD8⁺T cells are considered as major effector cells capable of controlling tumor growth, autologous CD8⁺T cells were adoptively transferred in OncoHumice bearing breast cancer tumors. These tumors completely regressed with time (FIG. 4a). As expected control tumors that received PBS injections progressed (FIG. 4a and 4b). To establish whether clearance of established breast cancer tumors by adoptively transferred CD8⁺T cells depended on the presence of DCs, purified CD8⁺T cells were administered into to breast cancer tumors in OncoMouse, i.e., mouse without human DCs. As shown in FIG. 4c, in the absence of DCs, breast cancer tumors were not cleared. These results suggest that indirect antigen presentation had occurred and that DCs are functional in vivo and cross-prime CD8⁺T cell immunity.

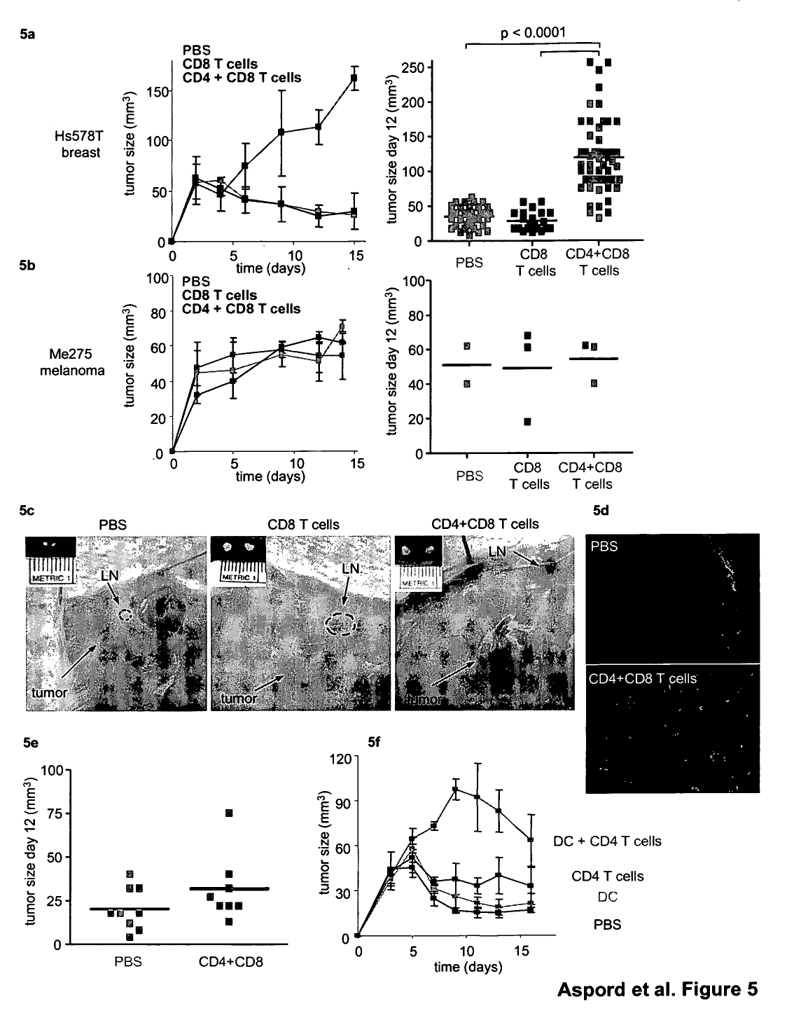

It was next determined whether CD4⁺T cells would help in the clearance of established tumors¹⁹⁻²¹. Reconstitution of Hs587T breast cancer tumors (FIG. 5a), but not Me275 melanoma (FIG. 5b) with CD4⁺T cells was associated with accelerated tumor development and increased primary tumor mass. CD4⁺T cells also accelerated the development of MCF7 breast cancer tumors (FIG. 5g). To determine the pathology of accelerated tumor development, mice were sacrificed at day 15^{th} and both the breast cancer tumors and their draining lymph nodes were subjected to macroscopic and microscopic analysis. Macroscopic analysis of Hs578T breast cancer tumors injected with PBS demonstrated a clearly visible tumor and a small draining lymph node (FIG. 5c). The tumors in mice injected with CD8⁺T cells were barely visible and the draining lymph nodes were enlarged consistent with an ongoing immune reaction (FIG. 5c). Strikingly, the tumors of mice injected with CD4⁺T cells were enlarged; both the tumor and the draining lymph node were inflamed with signs of enhanced vascularization (FIG. 5c). Microscopic analysis confirmed this pattern. Indeed, tumors with CD4⁺T cells demonstrated considerably enhanced vascularization visualized by the capture of FITC-lectin (FIG. 5d).

To determine whether accelerated development of breast cancer tumors was dependent on tumor infiltrating DCs, OncoMice with breast cancer tumors were administered CD4⁺T cells. Tumor development was not accelerated unless the mice were reconstituted with both CD4⁺T cells and DCs (FIGS. 5e and f). These results indicate that DCs are necessary for CD4⁺T cells to induce acceleration of breast tumor development.

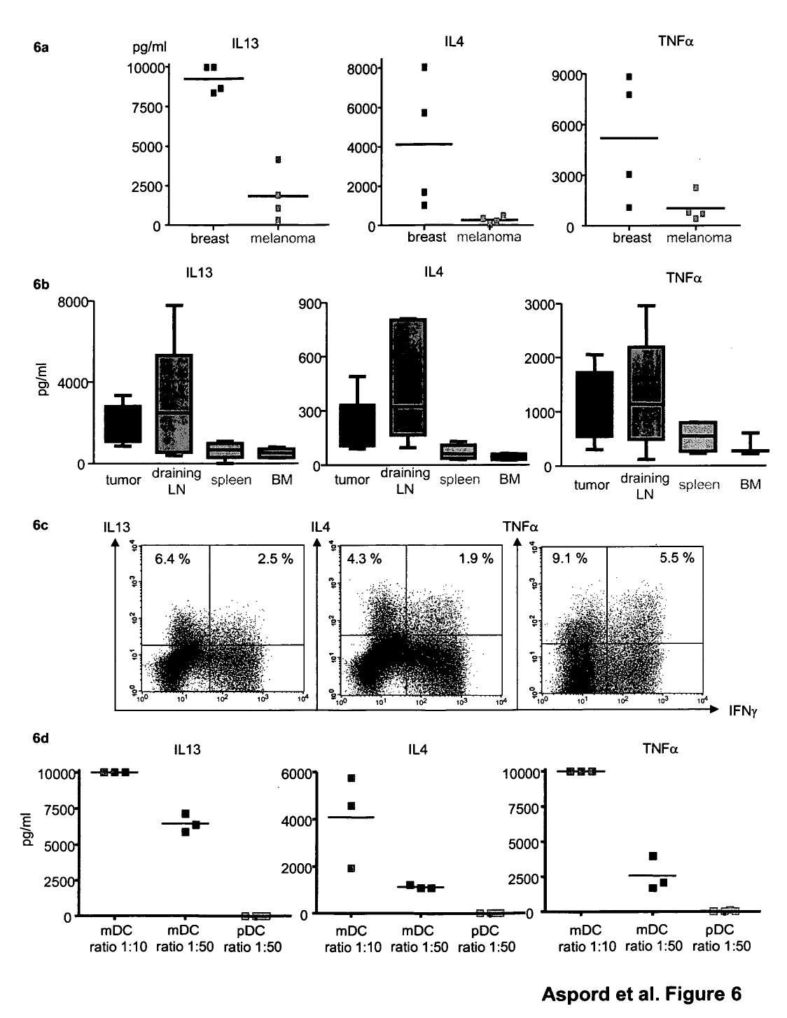

Breast cancer tumor-derived DCs prime IL-13 secreting CD4⁺T cells. To determine the mechanism by which CD4⁺T cells accelerate breast cancer tumor development, it was determined breast cancer tumor-derived DCs skew differentiation of naive CD4⁺T cells. DCs were sorted from tumor, draining lymph nodes, spleen and bone marrow 4 and 30 days after. breast cancer or melanoma inoculation. Naive allogeneic CD4⁺T cells were exposed for 5 days to sorted DCs and cytokines were assessed in the supernatants after restimulation with PMA and lonomycin. DCs isolated from lymph nodes draining either breast cancer tumors or melanoma tumors induced allogeneic CD4⁺T cells to proliferate and to secrete large amounts (>10 ng/ml) of IL-2 and IFN gamma (not shown). However, DCs isolated from the lymph nodes draining breast cancer tumors primed CD4⁺T cells to secrete significantly higher levels of IL-4, IL-13 and TNF (FIG. 6a) than DCs isolated from lymph nodes draining melanoma tumors. Thus, the mean concentration of IL-13 in the cocultures with DCs derived from lymph nodes draining breast cancer tumors was 9261±430 while it was 1848±828 in cocultures with DCs draining melanoma tumors (p=0.002). In OncoHumice bearing breast cancer tumors, the capacity to prime IL-4, IL-13 and TNF secreting CD4⁺T cells was found predominantly in DCs isolated from breast cancer tumors and their draining lymph nodes (FIG. 6b). Intracellular cytokine staining confirmed that IL-13, IL-4 and TNF are expressed by CD4⁺T cells only (FIG. 6c). Approximately 10% of CD4⁺T cells express IL-13 suggesting that a fraction of CD4⁺T cells can secrete large amounts of IL-13 and TNF. These results demonstrated that breast cancer polarizes DCs in such fashion that they prime CD4⁺T cells to produce type 2 and proinflammatory cytokines.

NKT cells were shown to secrete IL-13 and to block tumor-specific CD8⁺T cells in a mouse model of tumor²². Yet, an expansion of Vα24⁺ Vβ11⁺ CD4⁺T cells was not observed and depleting naive CD4⁺T cells of Vα24⁺ cells did not abolish IL-13 secretion (data not shown). While these results do not exclude the potential involvement of NKT cells, they indicate that the conventional CD4⁺T cells are the major source of IL-13 in this model.

As both pDCs and mDCs have been shown to be able to induce either Type 1 or Type 2 CD4⁺T cell immunity⁴, each DC subset was sorted and cultured with naive allogeneic CD4⁺T cells. As shown in FIG. 6d, the induction of CD4⁺T cells secreting IL-4, IL-5, IL-13 and TNF was unique to mDCs. Thus, breast cancer cells target mDCs and polarize them to induce Th2 and proinflammatory immunity.

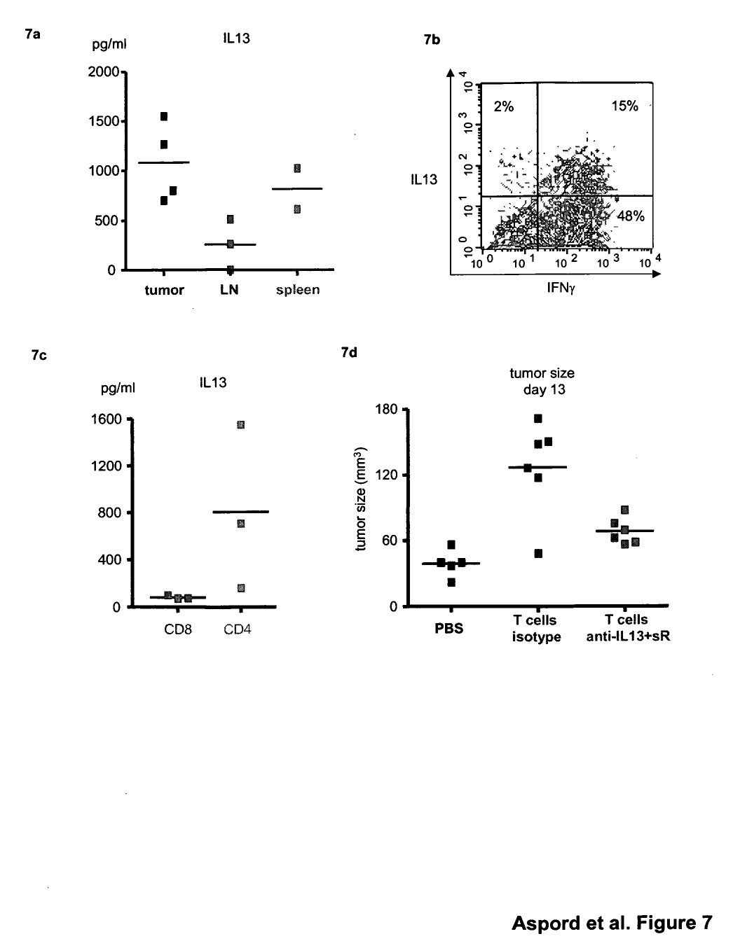

IL-13 mediates accelerated development of breast cancer tumors. Earlier studies pointed out IL-13 as a tumorigenic factor. The inventors determined IL-13's role in the CD4⁺T cell dependent acceleration of breast cancer tumor development. As shown in FIG. 7a, in 4/4 analyzed mice CD4⁺T cells isolated from breast cancer tumor showed high IL-13 secretion, i.e., up to 1500 pg/ml IL-13. Intracytoplasmic staining and flow cytometry demonstrate that IL-13 expressing CD4⁺T cells could be detected at a high frequency (10-15%) in the tumor bed (FIG. 7b). These results were further confirmed in an independent cohort of OncoHumice, where CD4⁺T cells, but not CD8⁺T cells, isolated from breast cancer tumor bed secreted considerable amounts of IL-13 (up to 1600 pg/ml) (FIG. 7c).

To determine whether IL-13 antagonists would prevent the accelerated tumor development, OncoHumice with Hs578T breast cancer tumors and autologous CD4⁺ and CD8⁺T cells received either a combination of antibodies blocking human IL-13 and IL13R (three times over 6 days) or isotype control. As expected, control mice demonstrated a significant increase in tumors mass at day 14^{th} post T-cell transfer (FIG. 7d). OncoHumice treated with IL-13 antagonists showed significant (p=0.02) decrease in the tumor mass (FIG. 7d). These results demonstrated the role of IL-13 in accelerated breast cancer tumor development in this model.

The model disclosed herein allows the study of distinct interactions between the different human cancers and the human immune system in vivo. Indeed, a novel pathway used by breast cancer, but not by melanoma, was found to promote its survival and development in the host. There, breast cancer tumors are heavily infiltrated with myeloid DCs which are then induced to mature into. a state that promotes the expansion of IL-13 secreting CD4⁺T cells. IL-13 in turn accelerates tumor development with rapid evolution of the tumor mass associated with massive inflammation. This picture resembles Inflammatory Breast Cancer, a clinical entity of unknown pathogenesis that represents an aggressive and distinct form of locally advanced breast cancer with a 5-year disease-free survival <50%. In this model, IL-13 accelerates tumor development possibly through non-immune mediated mechanisms including enhanced tumor vascularization. Indeed, the regulatory effect of IL-13 occurs at the very early phase of tumor development and may therefore be unrelated to the inhibition of CD8⁺T cell effector function. Accordingly, these results suggest that in vivo CTL priming is not inhibited by the presence of CD4⁺T cells.

Furthermore, this model permits the dissection of the critical steps of tumor-mediated manipulation of immune cells thereby providing novel therapeutic targets. There, the therapeutic targets are not tumor cells themselves, as in the classical chemotherapy or even immunotherapy approaches, but the tumor microenvironment. Furthermore, rather than targeting tumor stromal cells and/or tumor vasculature this model permits to target tumor infiltrating immune cells that might be held responsible for the enhanced vascularization. Thus, the first step towards new therapies could be the manipulation of DCs trafficking to tumor which in the case of breast cancer could be inhibited using specific agonists once the molecules governing DC attraction would have been identified. That might be important at two phases of this immune response, i.e., at the induction phase when the undesirable CD4+T cell immunity is induced and at the effector phase where the primed CD4+T cells arriving at the tumor site interact with tumor infiltrating DCs. Along with DC attraction, targeting the molecules responsible for the polarization of DC function could help prevent the development of pro-cancer CD4+T cells. These results show that the latter arriving cells act at least partially via enhanced vascularization, this would represent a novel approach to anti-angiogenic therapy.

Thus, the model can be used at both basic and clinical level. At the basic level it will permit to determine the mechanisms tumors use to escape the immune system and to identify molecules the targeting of which might be used for therapy. At the clinical level the OncoHumouse will eventually permit us to design strategies to eliminate tumor cells through the manipulation of the immune system such as vaccination, antibody therapy, and adoptive transfer coupled or not to traditional chemotherapy regimens.

TABLE 3
Human Tumor Cell Lines

| CELL LINE | TUMOR TYPE |
|---|---|
| J82 | Transitional-cell carcinoma, bladder |
| RT4 | Transitional-cell papilloma, bladder |
| ScaBER | Squamous carcinoma, bladder |
| T24 | Transitional-cell carcinoma, bladder |
| TCCSUP | Transitional-cell carcinoma, bladder, primary |
| | grade IV |
| 5637 | Carcinoma, bladder, primary |
| SK-N-MC | Neuroblastoma, metastasis to supra-orbital area |
| SK-N-SH | Neuroblastoma, mietastasis to bone marrow |
| SW 1088 | Astrocytoma |
| SW 1783 | Astrocytoma |
| U-87 MG | Glioblastoma, astrocytoma, grade III |
| U-118 MG | Glioblastoma |
| U-138 MG | Glioblastoma |
| U-373 MG | Glioblastoma, astrocytoma, grade III |
| Y79 | Retinoblastoma |
| BT-20 | Carcinoma, breast |
| BT-474 | Ductal carcinoma, breast |
| MCF7 | Breast adenocarcinoma, pleural effusion |
| MDA-MB-134-V | Breast, ductal carcinoma, pleural I effusion |
| MDA-MD-157 | Breast medulla, carcinoma, pleural effusion |
| MDA-MB-175-VII | Breast, ductal carcinoma, pleural effusion |
| MDA-MB-361 | Adenocarcinoma, breast, metastasis to brain |
| SK-BR-3 | Adenocarcinoma, breast, malignant pleural effusion |
| C-33 A | Carcinoma, cervix |
| HT-3 | Carcinoma, cervix, metastasis to lymph node |
| ME-180 | Epidermoid carcinoma, cervix, metastasis to |
| | omentum |
| MS751 | Epidermoid carcinoma, cervix, metastasis to |
| | lymph node |
| SiHa | Squamous carcinoma, cervix |
| JEG-3 | Choriocarcinoma |
| Caco-2 | Adenocarcinoma, colon |
| HT-29 | Adenocarcinoma, colon, moderately well- |
| | differentiated grade II |
| SK-CO-1 | Adenocarcinoma, colon, ascites |
| HuTu 80 | Adenocarcinoma, duodenum |
| A-253 | Epidermoid carcinoma, submaxillary gland |
| FaDu | Squamous cell carcinoma, pharynx |
| A-498 | Carcinoma, kidney |
| A-704 | Adenocarcinoma, kidney |
| Caki-1 | Clear cell carcinoma, consistent with renal primary, |
| | metastasis to skin |
| Caki-2 | Clear cell carcinoma, consistent with renal primary |
| SK-NEP-1 | Wilms' tumor, pleural effusion |
| SW 839 | Adenocarcinoma, kidney |
| SK-HEP-1 | Adenocarcinoma, liver, ascites |
| A-427 | Carcinoma, lung |
| Calu-1 | Epidermoid carcinoma grade III, lung, metastasis to |
| | pleura |
| Calu-3 | Adenocarcinoma, lung, pleural effusion |
| Calu-6 | Anaplastic carcinoma, probably lung |
| SK-LU-1 | Adenocarcinoma, lung consistent with poorly |
| | differentiated, grade III |
| SK-MES-1 | Squamous carcinoma, lung, pleural effusion |
| SW 900 | Squamous cell carcinoma, lung |
| EB1 | Burkitt lymphoma, upper maxilia |
| EB2 | Burkitt lymphoma, ovary |
| P3HR-1 | Burkitt lymphoma, ascites |
| HT-144 | Malignant melanoma, metastasis to subcutaneous |
| | tissue |
| Malme-3M | Malignant melanoma, metastasis to lung |
| RPMI-7951 | Malignant melanoma, metastasis to lymph node |
| SK-MEL-1 | Malignant melanoma, metastasis to lymphatic |
| | system |
| SK-MEL-2 | Malignant melanoma, metastasis to skin of thigh |
| SK-MEL-3 | Malignant melanoma, metastasis to lymph node |
| SK-MEL-5 | Malignant melanoma, metastasis to axillary node |
| SK-MEL-24 | Malignant melanoma, metastasis to node |
| SK-MEL-28 | Malignant melanoma |
| SK-MEL-31 | Malignant melanoma |
| Caov-3 | Adenocarcinoma, ovary, consistent with primary |
| Caov-4 | Adenocarcinoma, ovary, metastasis to subserosa of |
| | fallopian tube |
| SK-OV-3 | Adenocarcinoma, ovary, malignant ascites |
| SW 626 | Adenocarcinoma, ovary |
| Capan-1 | Adenocarcinoma, pancreas, metastasis to liver |
| Capan-2 | Adenocarcinoma, pancrease |
| DU 145 | Carcinoma, prostate, metastasis to brain |
| A-204 | Rhabdomyosarcoma |
| Saos-2 | Osteogenic sarcoma, primary |
| SK-ES-1 | Anaplastic osteosarcoma versus Swing sarcoma, |
| | bone |
| SK-LNS-1 | Leiomyosarcoma, vulva, primary |
| SW 684 | Fibrosarcoma |
| SW 872 | Liposarcoma |
| SW 982 | Axilla synovial sarcoma |
| SW 1353 | Chondrosarcoma, humerus |
| U-2 OS | Osteogenic sarcoma, bone primary |
| Malme-3 | Skin fibroblast |
| KATO III | Gastric carcinoma |
| Cate-1B | Embryonal carcinoma, testis, metastasis to lymph |
| | node |
| Tera-1 | Embryonal carcinoma, malignancy consistent with |
| | metastasis to lung |
| Tera-2 | Embryonal carcinoma, malignancy consistent with, |
| | metastasis to lung |
| SW579 | Thyroid carcinoma |
| AN3 CA | Endometrial adenocarcinoma, metastatic |
| HEC-1-A | Endometrial adenocarcinoma |
| HEC-1-B | Endometrial adenocarcinoma |
| SK-UT-1 | Uterine, mixed mesodermal tumor, consistent with |
| | leiomyosarcoma grade III |
| SK-UT-1B | Uterine, mixed mesodermal tumor, consistent with |
| | leiomyosarcoma grade III |
| SW 954 | Squamous cell carcinoma, vulva |
| SW 962 | Carcinoma, vulva, lymph node metastasis |
| NCI-H69 | Small cell carcinoma, lung |
| NCI-H128 | Small cell carcinoma, lung |
| BT-483 | Ductal carcinoma, breast |
| BT-549 | Ductal carcinoma, breast |
| DU4475 | Metastatic cutaneous nodule, breast carcinoma |
| HBL-100 | Breast |
| Hs 578Bst | Breast, normal |
| Hs 578T | Ductal carcinoma, breast |
| MDA-MB-330 | Carcinoma, breast |
| MDA-MB-415 | Adenocarcinoma, breast |
| MDA-MB-435S | Ductal carcinoma, breast |
| MDA-MB-436 | Adenocarcinoma, breast |
| MDA-MB-453 | Carcinoma, breast |
| MDA-MB-468 | Adenocarcinoma, breast |
| T-47D | Ductal carcinoma, breast, pleural effusion |
| Hs 766T | Carcinoma, pancreas, metastatic to lymph node |
| Hs 746T | Carcinoma, stomach, metastatic to left leg |
| Hs 695T | Amelanotic melanoma, metastatic to lymph node |
| Hs 683 | Glioma |
| Hs 294T | Melanoma, metastatic to lymph node |
| Hs 602 | Lymphoma, cervical |
| JAR | Choriocarcinoma, placenta |
| Hs 445 | Lymphoid, Hodgkin's disease |
| Hs 700T | Adenocarcinoma, metastatic to pelvis |
| H4 | Neuroglioma, brain |
| Hs 696 | Adenocarcinoma primary, unknown, metastatic to |
| | bone-sacrum |
| Hs 913T | Fibrosarcoma, metastatic to lung |
| Hs 729 | Rhabdomyosarcoma, left leg |
| FHs 738Lu | Lung, normal fetus |
| FHs 173We | Whole embryo, normal |
| FHs 738Bl | Bladder, normal fetus |
| NIH: OVCAR-3 | Ovary, adenocarcinoma |
| Hs 67 | Thymus, normal |
| RD-ES | Ewing's sarcoma |
| ChaGo K-1 | Bronchogenic carcinoma, subcutaneous metastasis, |
| | human |
| WERI-Rb-1 | Retinoblastoma |
| NCI-H446 | Small cell carcinoma, lung |
| NCI-H209 | Small cell carcinoma, lung |
| NCI-H146 | Small cell carcinoma, lung |
| NCI-H441 | Papillary adenocarcinoma, lung |
| NCI-H82 | Small cell carcinoma, lung |
| H9 | T-cell lymphoma |
| NCI-H460 | Large cell carcinoma, lung |
| NCI-H596 | Adenosquamous carcinoma, lung |
| NCI-H676B | Adenocarcinoma, lung |
| NCI-H345 | Small cell carcinoma, lung |
| NCI-H820 | Papillary adenocarcinoma, lung |
| NCI-H520 | Squamous cell carcinoma, lung |
| NCI-H661 | Large cell carcinoma, lung |
| NCI-H510A | Small cell carcinoma, extra-pulmonary origin, |
| | metastatic |
| D283 Med | Medulloblastoma |
| Daoy | Medulloblastoma |
| D341 Med | Medulloblastoiua |
| AML-193 | Acute monocyte leukemia |
| MV4-11 | Leukemia biphenotype |

One or more primary cancer cells (or cancer cell lines derived therefrom) may be implanted in the Oncohumouse, e.g., those selected from lung, breast, melanoma, colon, renal, testicular, ovarian, lung, prostate, hepatic, germ cancer, epithelial, prostate, head and neck, pancreatic cancer, glioblastoma, astrocytoma, oligodendroglioma, ependymomas, neurofibrosarcoma, meningia, liver, spleen, lymph node, small intestine, blood cells, colon, stomach, thyroid, endometrium, prostate, skin, esophagus, bone marrow and blood.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

1. Foley, E. J. Antigenic properties of methylcholanthrene-induced tumors in mice of the strain of origin. Cancer Res 13, 835-7 (1953).

2. Klein, G., Sjogren, H. O., Klein, E. & Hellstrom, K. E. Demonstration of resistance against methylcholanthrene-induced sarcomas in the primary autochthonous host. Cancer Res 20, 1561-72 (1960).

3. Mestas, J. & Hughes, C. C. Of mice and not men: differences between mouse and human immunology. J Immunol 172, 2731-8 (2004).

4. Shortman, K. & Liu, Y. J. Mouse and human dendritic cell subtypes. Nature Rev Immunol 2, 151-61 (2002).

5. Brigl, M. & Brenner, M. B. CD1: antigen presentation and T cell function. Annu Rev Immunol 22, 817-90 (2004).

6. Cerwenka, A. & Lanier, L. L. Natural killer cells, viruses and cancer. Nat Rev Immunol 1, 41-9 (2001).

7. Palucka, A. K. et al. Human dendritic cell subsets in NOD/SCID mice engrafted with CD34⁺ hematopoietic progenitors. Blood 102, 3302-10 (2003).

8. Nagorsen, D., Scheibenbogen, C., Marincola, F. M., Letsch, A. & Keilholz, U. Natural T cell immunity against cancer. Clin Cancer Res 9, 4296-303 (2003).

9. Romero, P. et al. Ex vivo staining of metastatic lymph nodes by class I major histocompatibility complex tetramers reveals high numbers of antigen-experienced tumor-specific cytolytic T lymphocytes. J Exp Med 188, 1641-50 (1998).

10. Albert, M. L. et al. Tumor-specific killer cells in paraneoplastic cerebellar degeneration. Nat Med 4, 1321-4 (1998).

11. Disis, M. L. & Cheever, M. A. Oncogenic proteins as tumor antigens. Curr Opin Immunol 8, 637-42 (1996).

12. Coronella-Wood, J. A. & Hersh, E. M. Naturally occurring B-cell responses to breast cancer. Cancer Immunol Immunother 52, 715-38 (2003).

13. Scanlan, M. J. et al. Humoral immunity to human breast cancer: antigen definition and quantitative analysis of mRNA expression. Cancer Immun 1, 4 (2001).

14. Darnell, J. C., Albert, M. L. & Darnell, R. B. Cdr2, a target antigen of naturally occuring human tumor immunity, is widely expressed in gynecological tumors. Cancer Res 60, 2136-9 (2000).

15. Bell, D. et al. In breast carcinoma tissue, immature dendritic cells reside within the tumor, whereas mature dendritic cells are located in peritumoral areas. J Exp Med 190, 1417-26 (1999).

16. Vermi, W. et al. Recruitment of immature plasmacytoid dendritic cells (plasmacytoid monocytes) and myeloid dendritic cells in primary cutaneous melanomas. J Pathol 200, 255-68 (2003).

17. Enk, A. H., Jonuleit, H., Saloga, J. & Knop, J. Dendritic cells as mediators of tumor-induced tolerance in metastatic melanoma. Int J Cancer 73, 309-16 (1997).

18. Lee, J. R. et al. Pattern of recruitment of immunoregulatory antigen-presenting cells in malignant melanoma. Lab Invest 83, 1457-66 (2003).

19. Sun, J. C. & Bevan, M. J. Defective CD8 T cell memory following acute infection without CD4 T cell help. Science 300, 339-42 (2003).

20. Shedlock, D. J. & Shen, H. Requirement for CD4 T cell help in generating functional CD8 T cell memory. Science 300, 337-9 (2003).

21. Janssen, E. M. et al. CD4+ T cells are required for secondary expansion and memory in CD8+ T lymphocytes. Nature 421, 852-6 (2003).

22. Terabe, M. et al. NKT cell-mediated repression of tumor immunosurveillance by IL-13 and the IL-4R-STAT6 pathway. Nat Immunol 1, 515-20 (2000).

23. Muller, A. et al. Involvement of chemokine receptors in breast cancer metastasis. Nature 410, 50-6 (2001).

24. Palucka, A. K. et al. Acute lymphoblastic leukemias from relapse engraft more rapidly in SCID mice. Leukemia 10, 558-63 (1996).

25. Bankert, R. B., Egilmez, N. K. & Hess, S. D. Human-SCID mouse chimeric models for the evaluation of anti-cancer therapies. Trends Immunol 22, 386-93 (2001).

26. de Bont, E. S. et al. Mobilized human CD34⁺ hematopoietic stem cells enhance tumor growth in a nonobese diabetic/severe combined immunodeficient mouse model of human non-Hodgkin's lymphoma. Cancer Res 61, 7654-9 (2001).

27. Nguyen, T., Naziruddin, B., Dintzis, S., Doherty, G. M. & Mohanakumar, T. Recognition of breast cancer-associated peptides by tumor-reactive, HLA-class I restricted allogeneic cytotoxic T lymphocytes. Int J Cancer 81, 607-15 (1999).

28. Lumkul, R. et al. Human AML cells in NOD/SCID mice: engraftment potential and gene expression. Leukemia 16, 1818-26 (2002).

What is claimed is:

1. An in vivo model for the study of the human immune system comprising:
an immune deficient mouse made chimeric with one or more human hematopoietic progenitor cells and one or more human tumor cell lines.

2. The model of claim 1, wherein the immune deficient mouse comprises a immunodeficient nonobese diabetic/LtSz-scid/scid (NOD/SCID).

3. The model of claim 1, wherein the immune deficient mouse comprises a NOD/SCID β-2 microglobulin^{-/-} mice.

4. The model of claim 1, wherein the human hematopoietic progenitor cells comprise CD34⁺ hematopoietic progenitors.

5. The model of claim 1, wherein the human hematopoietic progenitor cells comprise CD34⁺ hematopoietic progenitor cells contacted with G-CSF.

6. The model of claim 1, wherein the human hematopoietic progenitor cells comprise human DCs, myeloid cells and B cells.

7. The model of claim 1, wherein the human hematopoietic progenitor cells comprise autologous T cells.

8. The model of claim 1, wherein the human hematopoietic progenitor cells comprise human T cell subsets.

9. The model of claim 1, wherein the human tumor cell lines comprise breast cancer adenocarcinoma.

10. The model of claim 1, wherein the human tumor cell lines comprise malignant melanoma.

11. The model of claim 1, wherein the human tumor cell lines comprise Hs578T, MCF7, 1806, Me275, Sk-Mel24, COLO829 and combinations thereof.

12. The model of claim 1, wherein the immune deficient mouse is sub-lethally irradiated.

13. A pre-clinical in vivo model for the study of the interaction of human cancer with the human immune system comprising:
an immune deficient mouse made chimeric with one or more human hematopoietic progenitor cells and one or more human tumor cell lines.

14. A nonhuman mammal comprising an immune deficient mammal with one or more human hematopoietic progenitor cells and one or more human tumor cell lines.

15. The mammal of claim 13, wherein the mammal is a mouse, rat, rabbit, goat, pig.

16. The mammal of claim 13, wherein the cell is a tumor cell listed in Table 3.

17. A method for evaluating a test compound suspected of promoting or inhibiting cancer comprising administering to a test mammal comprising an immune deficient mouse made chimeric with one or more human hematopoietic progenitor cells and one or more human tumor cell lines with the test compound and monitoring the state of the one or more human tumor cell lines, a human immune response or both.

18. The method of claim 17, wherein the compound affects immune cells, cancer cells, vascular cells, stromal cells, and combinations thereof.

19. The method of claim 17, further comprising the step of evaluating a group of compounds to identify one or more lead compounds.

20. A method of identifying one or more lead compounds comprising the steps of:
administering to a chimeric immune deficient mouse comprising one or more human hematopoietic progenitor cells and one or more human tumor cell lines one or more members of a pool of test compounds; and
monitoring the state of the one or more human tumor cell lines, a human immune response or both, wherein one or more lead compounds are identified from the pool of test compounds.

21. The method of claim 17, further comprising the step of further reducing the size of the one or more pools to identify one or more specific families of compounds that affect the human immune cells, the human cancer cells, or both.

22. A pool of test compounds isolated and purified by a method comprising the steps of:
administering to a chimeric immune deficient mouse comprising one or more human hematopoietic progenitor cells and one or more human tumor cell lines one or more members of a pool of test compounds; and
monitoring the state of the one or more human tumor cell lines, the one or more human immune response or both, wherein one or more pools of lead compounds are identified from the pool of test compounds.

23. The method of claim 22, wherein the compound affects immune cells, cancer cells, vascular cells, stromal cells, and combinations thereof.

24. The method of claim 22, wherein the mouse further comprises one or more transgenic genes.

25. The method of claim 22, wherein the one or more human hematopoietic progenitor cells, the one or more human tumor cell lines, or both, further comprises one or more transgenic genes.

26. A compound identified by the method of claim 20.

27. A method for customizing a patient immune response comprising the steps of:
making one or more chimeric oncohumice with one or more tumor cells and one or more immune cells;
providing the one or more oncohumice with one or more agents that modulate the immune response; and
determining the effects of the immune response against the tumor cells after exposure to the one or more agents.

28. The method of claim 27, wherein the one or more agents comprise antigens, adjuvants, superantigens, lymphokines, chemokines, cytokines and combinations thereof.

29. The method of claim 27, wherein the patient immune cells, the tumor cells or both are autologous.

30. The method of claim 27, wherein the agents comprise a cocktail of agents that modulate immune responses toward a Th1 response, a Th2 response, an NK response, a CTL response, signaling through Toll receptors, T cell anergy or combinations thereof.
